(19) United States
(12) Patent Application Publication
(10) Pub. No.: US 2015/0073219 A1
(43) Pub. Date: Mar. 12, 2015

(54) STEREOSCOPIC ENDOSCOPE

(71) Applicant: SONY CORPORATION, Tokyo (JP)

(72) Inventors: Satoshi Nagae, Saitama (JP); Toshio Shirai, Kanagawa (JP)

(21) Appl. No.: 14/460,897

(22) Filed: Aug. 15, 2014

(30) Foreign Application Priority Data
Sep. 9, 2013 (JP) 2013-186481

(51) Int. Cl. A61B 1/00

(52) U.S. Cl. CPC A61B 1/00193
USPC 600/166

(57) ABSTRACT

A stereoscopic endoscope includes an imaging optical system including a diaphragm which adjusts light intensity and a pupil splitting polarizing element that has a pair of polarizers which are disposed to line up along a splitting line as a boundary; a scope holder in which at least the pupil splitting polarizing element is disposed in an inner portion thereof; and an imaging device on which light is incident via the imaging optical system. Light which is incident on the pupil splitting polarizing element is polarized by the pair of polarizers in order to generate a right eye image and a left eye image, respectively. A direction that is perpendicular to both the splitting line and an optical axis is a positioning direction. Positioning portions are provided to position the pupil splitting polarizing element in relation to a conjugate position of the diaphragm, or a proximity thereof, in the positioning direction.

### CROSS REFERENCE TO RELATED APPLICATIONS

This application claims the benefit of Japanese Priority Patent Application JP 2013-186481 filed Sep. 9, 2013, the entire contents of which are incorporated herein by reference.

### BACKGROUND

The present technology relates to a stereoscopic endoscope provided with a pupil splitting polarizing element that includes a pair of polarizers, in which light is polarized by a pair of polarizers in order to generate a right eye image and a left eye image, respectively, and the polarized light is incident on an imaging device.

A stereoscopic endoscope which uses an imaging optical system that acquires a stereoscopic image has been proposed in the related art (refer to Japanese Patent No. 3285217 and Japanese Unexamined Patent Application Publication No. 8-292379, for example).

The stereoscopic endoscope disclosed in Japanese Patent No. 3285217 is configured such that two lens barrels for a left eye and for a right eye, respectively, are disposed at an interval corresponding to a desired parallax amount, and that a stereoscopic image is obtained using the parallax between the left eye and the right eye.

The stereoscopic endoscope disclosed in Japanese Unexamined Patent Application Publication No. 8-292379 is configured such that a pupil splitting mirror which separates an optical path into two, for the left eye and for the right eye, is disposed at a pupil position of a single objective optical system, and that two sets of image forming optical systems which form two split images and corresponding two sets of imaging devices are disposed to acquire a stereoscopic image.

However, in the stereoscopic endoscope disclosed in Japanese Patent No. 3285217, since two lens barrels are necessary, the diameter is increased and the weight becomes heavy. In particular, in recent years, the frequency of performing surgical operations using a minimally invasive endoscope has increased; and, in such operations, an increase in the diameter of the portion that is inserted into the body entails an increase in the load on the patient.

In the stereoscopic endoscope disclosed in Japanese Unexamined Patent Application Publication No. 8-292379, a pupil splitting mirror, two sets of image forming optical systems, and two sets of imaging devices are necessary; as such, the number of components is great and the diameter and weight are increased.

Therefore, a type of stereoscopic endoscope in which a single imaging optical system and a single imaging device are disposed has been proposed as a stereoscopic endoscope in which an increase in the diameter and the weight is avoided and miniaturization and weight reduction are achieved (refer to Japanese Unexamined Patent Application Publication No. 2013-106189, for example).

The stereoscopic endoscope disclosed in Japanese Unexamined Patent Application Publication No. 2013-106189 is provided with a polarization filter that includes a first filter portion (a first region) and a second filter portion (a second region) for generating the right eye image and the left eye image, respectively. In the first filter portion, a first polarized light component which oscillates in a first direction is transmitted, and a second polarized light component which oscillates in a second direction, which is perpendicular to the first direction, is blocked. In the second filter portion, the first polarized light component is blocked, and the second polarized light component is transmitted. The first polarized light component, which is transmitted through the first filter portion, and the second polarized light component, which is transmitted through the second filter portion, are incident on an imaging device.

### SUMMARY

Incidentally, in the stereoscopic endoscope which is configured such that each polarization takes place at a different region, as disclosed in Japanese Unexamined Patent Application Publication No. 2013-106189, it is important for the acquisition of a favorable stereoscopic image that light of a predetermined intensity is incident on each region in which the polarization of the polarizer is performed, and it is necessary to secure high positional precision for each region.

It is desirable to acquire a favorable stereoscopic image by achieving an improvement in the positional precision of the pair of polarizers of the pupil splitting polarizing element.

According to an embodiment of the present technology, there is provided a stereoscopic endoscope, which includes an imaging optical system including a diaphragm which adjusts light intensity and a pupil splitting polarizing element that has a pair of polarizers which are disposed to line up along a splitting line as a boundary; a scope holder in which at least the pupil splitting polarizing element is disposed in an inner portion thereof; and an imaging device on which light is incident via the imaging optical system. Light which is incident on the pupil splitting polarizing element is polarized by the pair of polarizers in order to generate a right eye image and a left eye image, respectively. A direction that is perpendicular to both the splitting line and an optical axis is a positioning direction. Positioning portions are provided to position the pupil splitting polarizing element in relation to a conjugate position of the diaphragm, or a proximity thereof, in the positioning direction.

Accordingly, the pupil splitting polarizing element is positioned, by the positioning portion, in relation to the conjugate position of the diaphragm, or the proximity thereof, in the positioning direction.

It is desirable that the stereoscopic endoscope described above further include an element holder which holds the pupil splitting polarizing element.

Accordingly, the pupil splitting polarizing element is attached to the scope holder in a state of being held in the element holder.

In the stereoscopic endoscope described above, it is desirable that positioning grooves which extend in the positioning direction be formed in one of the scope holder and the element holder, that positioning pins which extend in an optical axis direction and are supported in the positioning grooves to slide freely be provided on the other of the scope holder or the element holder, that the positioning portions be configured of the positioning pins and the positioning grooves, and that the pupil splitting polarizing element be positioned by changes in relative position of the positioning pins and the positioning grooves.

Accordingly, the pupil splitting polarizing element is positioned due to the relative position between the positioning pin and the positioning groove changing when the positioning pin is guided by the positioning groove.

It is desirable that the stereoscopic endoscope described above further include adjusting screws of which a position thereof in the positioning direction is changed when distal ends thereof are pressed against the element holder and the adjusting screws are rotated, and that the positioning be performed when the adjusting screws are rotated, with positions of the element holder and the pupil splitting polarizing element being changed.

Accordingly, the position of the pupil splitting polarizing element changes according to the rotation amount of the adjusting screws.

In the stereoscopic endoscope described above, it is desirable that the adjusting screw be rotated by an adjusting jig, and that a jig insertion hole into which the adjusting jig is inserted be formed in the scope holder.

Accordingly, the positioning is performed when the adjusting screw is rotated by the adjusting jig from the outside of the scope holder.

In the stereoscopic endoscope described above, it is desirable that positioning marks for positioning the splitting line during attachment of the pupil splitting polarizing element to the element holder be formed in the element holder.

Accordingly, the pupil splitting polarizing element is positioned when the splitting line is matched to the positioning marks.

In the stereoscopic endoscope described above, it is desirable that a retaining surface be formed on the scope holder, and that biasing springs which bias the element holder in an optical axis direction, press the element holder against the retaining surface, and position the pupil splitting polarizing element in the optical axis direction.

Accordingly, the attachment of the pupil splitting polarizing element to the scope holder and the positioning of the pupil splitting polarizing element in the optical axis direction are performed at the same time.

In the stereoscopic endoscope described above, it is desirable that cut-out surfaces be formed on an outer circumference of the element holder, and that member disposition spaces, in which predetermined members are disposed, be formed between the element holder and the scope holder by the cut-out surfaces.

Accordingly, the space in the inner portion of the scope holder is used as a space for disposing predetermined members.

In the stereoscopic endoscope described above, it is desirable that the pupil splitting polarizing element be disposed in the conjugate position of the diaphragm.

Accordingly, the pupil splitting polarizing element is disposed in an optimal position without influencing the optical performance of the imaging optical system.

In the stereoscopic endoscope described above, it is desirable that the pupil splitting polarizing element be disposed in a proximity of the conjugate position of the diaphragm.

Accordingly, the pupil splitting polarizing element is disposed in an optimal position taking aberration which occurs in the imaging optical system into consideration.

In the stereoscopic endoscope of the embodiment of the present technology, the pupil splitting polarizing element is positioned, by the positioning portion, in relation to the conjugate position of the diaphragm, or the proximity thereof, in the positioning direction; thus it is possible to acquire a favorable stereoscopic image with an improvement in the positional precision of the pair of polarizers.

### BRIEF DESCRIPTION OF THE DRAWINGS

FIG. 1, together with FIGS. 2 to 22, shows a stereoscopic endoscope of an embodiment of the present technology, and is a perspective view of the stereoscopic endoscope;

FIG. 2 is a perspective view showing the stereoscopic endoscope in a state in which a monocular endoscope and an imaging head unit are separated;

FIG. 3 is an enlarged plan view of a scope holder;

FIG. 4 is a schematic view showing an imaging device;

FIG. 5 is an enlarged front view of the scope holder;

FIG. 6 is an enlarged vertical cross sectional view of the scope holder;

FIG. 7 is an enlarged horizontal cross sectional view of the scope holder;

FIG. 8 is a schematic enlarged exploded perspective view of a polarizing element block;

FIG. 9 is a schematic enlarged side view of the polarizing element block;

FIG. 10 is an enlarged perspective view of an element holder;

FIG. 11 is an enlarged front view of the element holder;

FIG. 12 is an enlarged horizontal cross sectional view of the element holder;

FIG. 13 is an enlarged perspective view of the polarizing element block;

FIG. 14 is a cross-sectional view along the XIV-XIV line of FIG. 13;

FIG. 15 is an enlarged exploded perspective view showing the scope holder, the polarizing element block, biasing springs and the like;

FIG. 16 is an enlarged perspective view showing a state in which the polarizing element block is disposed in an inner portion of the scope holder;

FIG. 17 is an enlarged perspective view showing the state in which the polarizing element block is disposed in the inner portion of the scope holder when viewed from a different direction from that in FIG. 16;

FIG. 18 is an enlarged horizontal cross sectional view showing a state in which the polarizing element block is disposed in the inner portion of the scope holder;

FIG. 19 is an enlarged vertical cross sectional view showing a state in which the polarizing element block is disposed in the inner portion of the scope holder;

FIG. 20 is an enlarged partial cross sectional front view showing a state in which the polarizing element block is disposed in the inner portion of the scope holder;

FIG. 21 is an enlarged partial cross sectional front view showing a state before positioning work of the polarizing element block in relation to the scope holder is performed; and

FIG. 22 is an enlarged partial cross sectional front view showing a state in which the positioning work of the polarizing element block in relation to the scope holder is performed.

### DETAILED DESCRIPTION OF EMBODIMENTS

Hereinafter, description will be given of an embodiment of the stereoscopic endoscope of the present technology according to the attached drawings.

In the description hereinafter, the directions front, rear, up, down, left and right are indicated as seen from the perspective of a photographer when photographing with the stereoscopic endoscope. Therefore, an object side is the front, and the side of the photographer (the image side) is the rear.

Note that, the directions front, rear, up, down, left and right indicated hereinafter are intended to facilitate explanation, and embodiments of the present technology are not limited to these directions.

### Schematic Configuration of Stereoscopic Endoscope

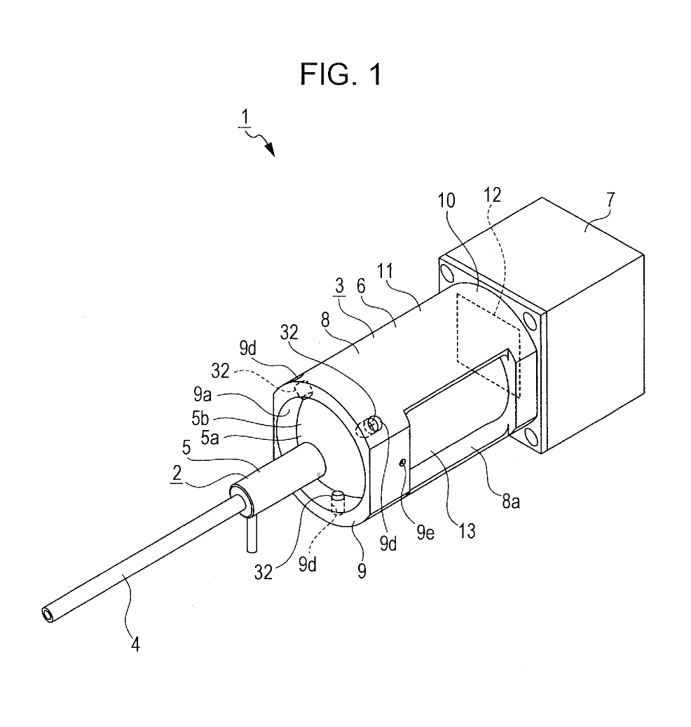

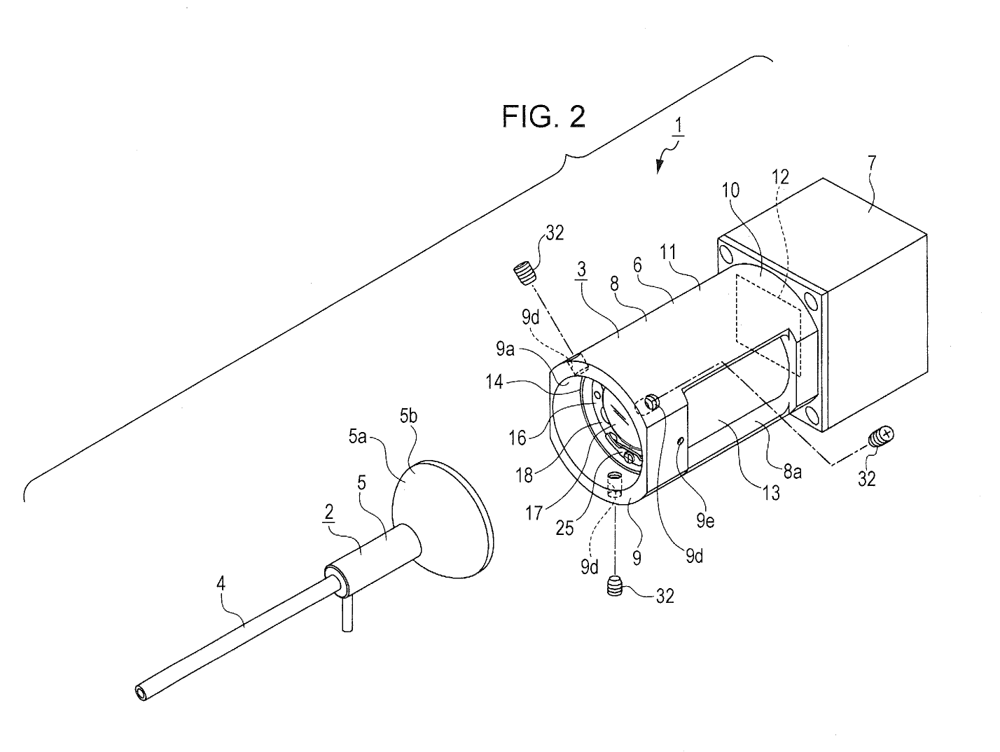

A stereoscopic endoscope 1 is configured of a removable monocular endoscope 2 and an imaging head unit 3 (refer to FIGS. 1 and 2). An imaging optical system that includes various optical components such as a lens, a diaphragm, and a polarizing element for imaging an object is provided in the inner portions of the monocular endoscope 2 and the imaging head unit 3.

The monocular endoscope 2 is a so-called hard mirror in which the portion that is inserted into the body is formed hard, and includes an insertion portion 4, which is long, thin and extends in the front-rear direction in which the insertion portion 4 is inserted into the body, and an observation portion 5 which is provided to continue from the rear end of the insertion portion 4. The diameter of the observation portion 5 is larger than that of the insertion portion 4.

The rear end portion of the observation portion 5 is provided as a joining portion 5a, which is formed in a shape in which the diameter thereof increases away from the insertion portion 4. The surface of the front side of the joining portion 5a is an inclined surface 5b.

An objective optical system (not shown) that includes a plurality of lenses and the like, generally one or more relay optical systems, and an ocular optical system are disposed in the inner portion of the monocular endoscope 2 in order from the front side of the insertion portion 4 to the rear side. A brightness diaphragm (hereinafter referred to as a “diaphragm”) is provided in the hard mirror optical system. An image of the object is formed by the objective optical system, and the formed image is transmitted to the ocular optical system by the relay optical system. During photography, the light that is incident on the monocular endoscope 2 from the object side forms a primary image between the objective optical system and the relay optical system, forms a secondary (or higher order) image between the relay optical system and the ocular optical system, and afocal light is emitted from the observation portion 5 toward the imaging head unit 3.

Note that, using the monocular endoscope 2, it is possible to perform monocular observation using a video system corresponding to the monocular endoscope 2, for example.

The imaging head unit 3 includes an imaging unit 6 and a connecting portion 7. The monocular endoscope 2 is joined to the front end portion of the imaging unit 6, and the connecting portion 7 is attached to the rear end portion of the imaging unit 6. A connector (not shown) is connected to the connecting portion 7, and the imaging head unit 3 is connected to a power source circuit or the like via the connector.

The imaging unit 6 is formed of a scope holder 8 that functions as an outer barrel, and the necessary components that are disposed in the inner portion of the scope holder 8.

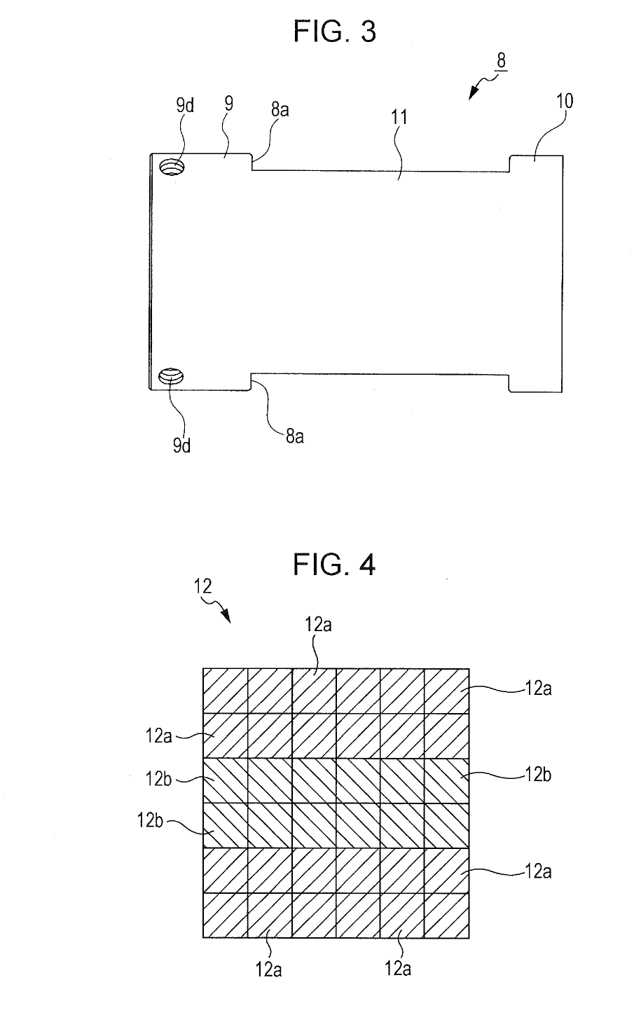

The scope holder 8 is formed in a shape that extends in the front-rear direction, and cutout portions 8a, 8a that are open to both sides are formed in a portion of the scope holder 8 excluding both the front and the rear end portions. In the scope holder 8, the front end portion and the rear end portion are each formed in a cylindrical shape, and as shown in FIG. 3, the front end portion is provided as an attaching portion 9, the rear end portion is provided as an element displacement portion 10, and the portion between both the front and the rear end portions is provided as a lens displacement portion 11.

An imaging device 12 is displaced on the element displacement portion 10 of the scope holder 8. As shown in FIG. 4, the imaging device 12 is a layered imaging device, and is configured such that wire-grid polarizers 12a, 12a, 12b, 12b, . . . of +45° and −45°, using a vertical line as a reference, are provided at predetermined corresponding positions on a detection surface.

An attachment lens 13 is disposed on the lens displacement portion 11 of the scope holder 8.

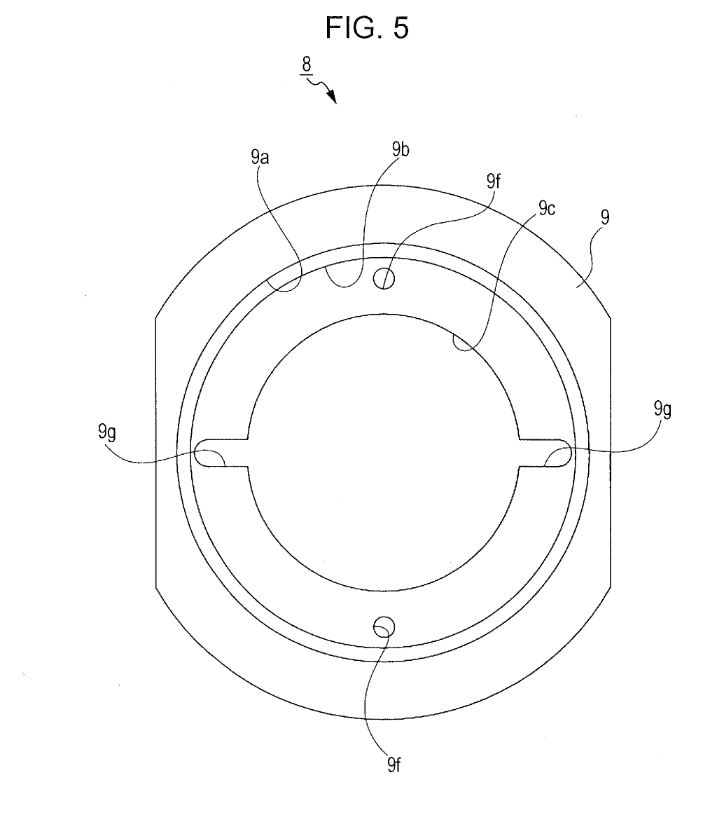

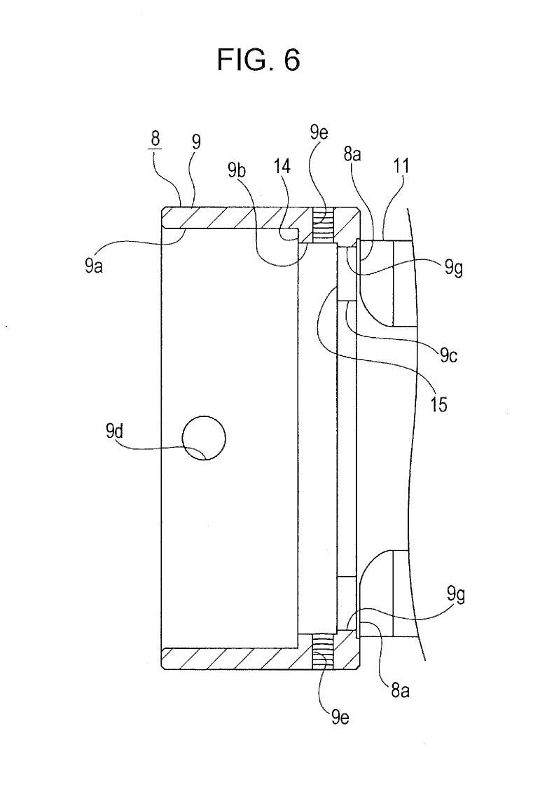

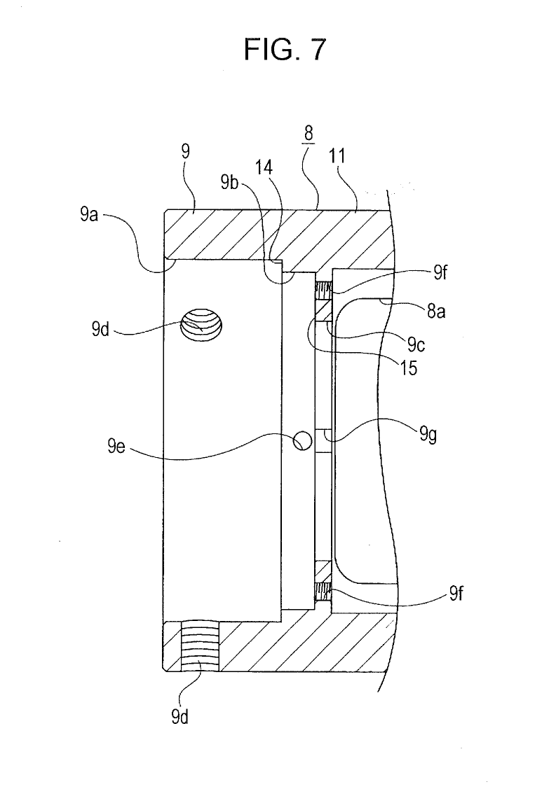

A joining space 9a, a disposition space 9b, and a communicating space 9c are formed in order from the front side on the inside of the attaching portion 9 of the scope holder 8 (refer to FIGS. 5 to 7). The outer diameters of the joining space 9a, the disposition space 9b, and the communicating space 9c become smaller in this order; and, in the inner circumferential portion of the attaching portion 9, a step-shaped bearing surface 14 facing forward is formed between the joining space 9a and the disposition space 9b, and a step-shaped retaining surface 15 facing forward is formed between the disposition space 9b and the communicating space 9c.

In the attaching portion 9, attaching holes 9d, 9d, 9d are formed on the portion on which the joining space 9a is formed to be spaced equidistantly from one another in the circumferential direction.

In the attaching portion 9, jig insertion holes 9e, 9e are formed on both left and right end portions, respectively, of the outside of the disposition space 9b, the jig insertion holes 9e, 9e are formed by penetrating to communicate the outside of the attaching portion 9 with the disposition space 9b. Screw grooves are formed in the jig insertion holes 9e, 9e.

In the attaching portion 9, screw holes 9f, 9f are formed on both top and bottom end portions, respectively, on the outside of the communicating space 9c, and the screw holes 9f, 9f are penetrated in the front-back direction. In the attaching portion 9, positioning grooves 9g, 9g which extend to the left and the right and are penetrated through the front and back are formed on both left and right end portions, respectively, on the outside of the communicating space 9c, and each of the positioning grooves 9g, 9g are open to the inside.

A polarizing element block 16 is displaced in the disposition space 9b of the attaching portion 9. The polarizing element block 16 is formed of a pupil splitting polarizing element 17, and an element holder 18 that holds the pupil splitting polarizing element 17.

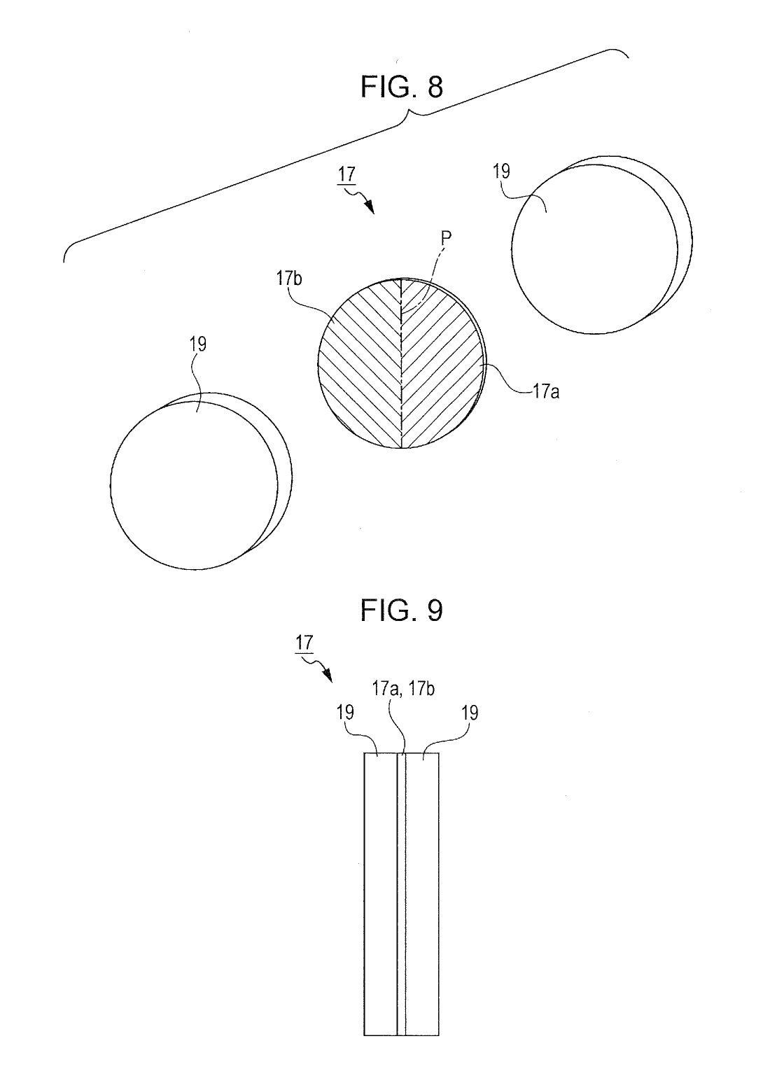

The pupil splitting polarizing element 17 includes a pair of polarizers 17a, 17b, each of which is formed in a crescent-shape, and the polarizers 17a, 17b are disposed to line up to the left and right with the linear side edge of each as the boundary (refer to FIG. 8). The boundary of the polarizers 17a, 17b is a splitting line P. Circular plate shaped glass plates 19, 19 are joined from the front and the rear to the polarizers 17a, 17b that are lined up on the left and the right (refer to FIGS. 8 and 9). The polarizers 17a, 17b have axes that easily transmit light polarized at +45° and at −45°, respectively, using the splitting line P as a reference. Note that, the transmission axes are not limited to those described above, and may also be 0° and 90°. In this case, the imaging device 12 is provided with wire-grid polarizers of 0° and 90°, using a vertical line as a reference.

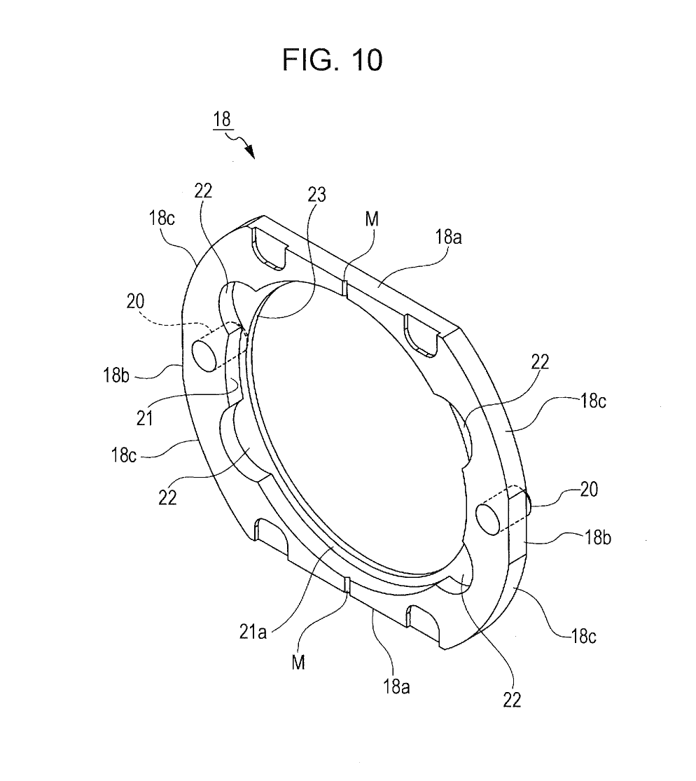

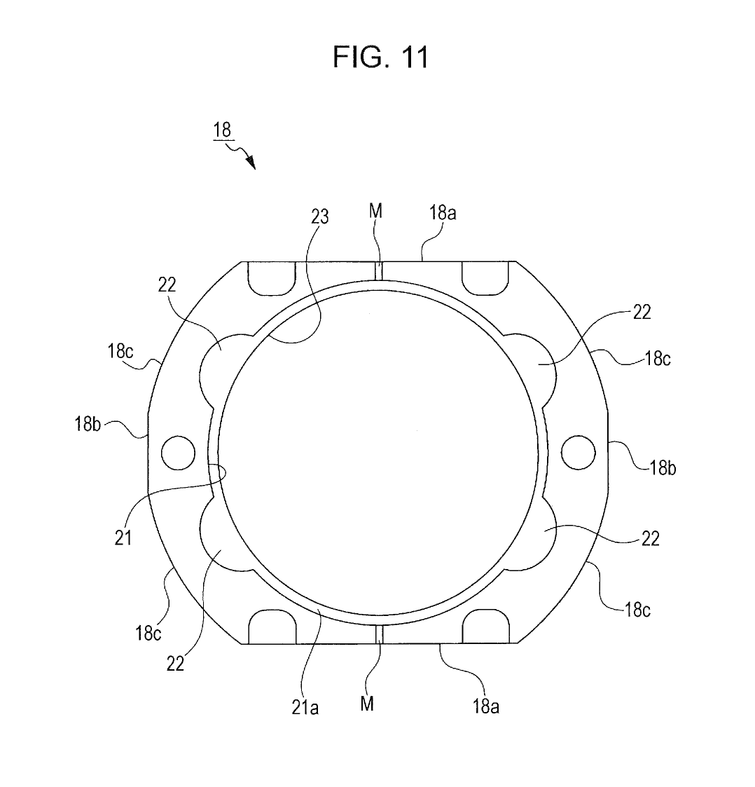

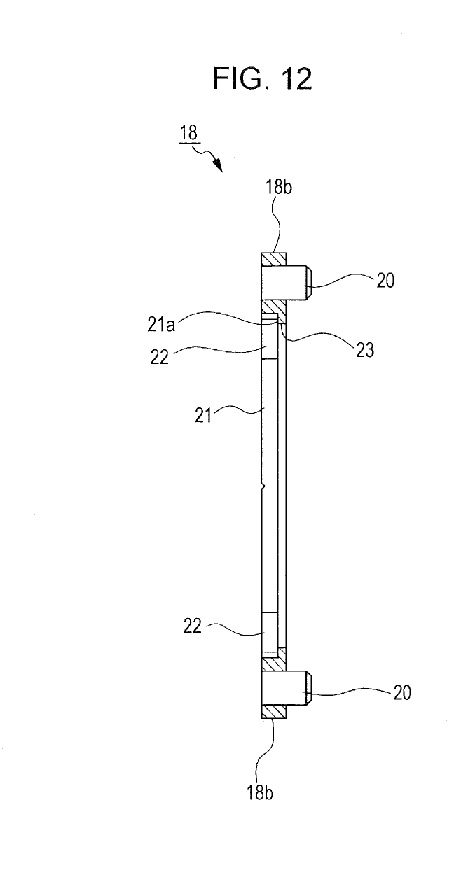

The element holder 18 is formed in an annular shape, and as shown in FIGS. 10 to 12, includes cutout surfaces 18a, 18a, side surfaces 18b, 18b, and four arc surfaces 18c, 18c, . . . . The cutout surfaces 18a, 18a are positioned on the top and bottom of the element holder 18 and the outer circumferential surfaces face upward or downward, the side surfaces 18b, 18b are positioned on the left and the right to face leftward or rightward, and the arc surfaces 18c, 18c, . . . extend in the circumferential direction. The four arc surfaces 18c, 18c, . . . are positioned between the cutout surfaces 18a, 18a and the side surfaces 18b, 18b, respectively. Positioning marks M, M that extend vertically are formed in the central portion in the horizontal direction of both top and bottom end portions on the front surface of the element holder 18.

Positioning pins 20, 20 are attached to both left and right end portions, respectively, of the element holder 18, and each of the positioning pins 20, 20 protrude to the rear. The diameter of the positioning pin 20 is formed at approximately the same size as the width in the vertical direction of the positioning groove 9g which is formed on the attaching portion 9 of the scope holder 8.

An attaching concave portion 21 which is open to the front is formed in the inner circumferential portion of the element holder 18. The outer diameter of the attaching concave portion 21 is approximately the same size as the outer diameter of the pupil splitting polarizing element 17. Adhesion concave portions 22, 22, . . . which are open to the front are formed in the outer circumferential side of the attaching concave portion 21 on the element holder 18 to be spaced from one another in the circumferential direction, and the adhesion concave portions 22, 22, continue from the attaching concave portion 21. The space of the rear side of the attaching concave portion 21 in the element holder 18 is formed as a light transmitting hole 23, and the diameter of the light transmitting hole 23 is smaller than the diameter of the attaching concave portion 21. The front surface that forms the attaching concave portion 21 of the element holder 18 is formed as a seating surface 21a.

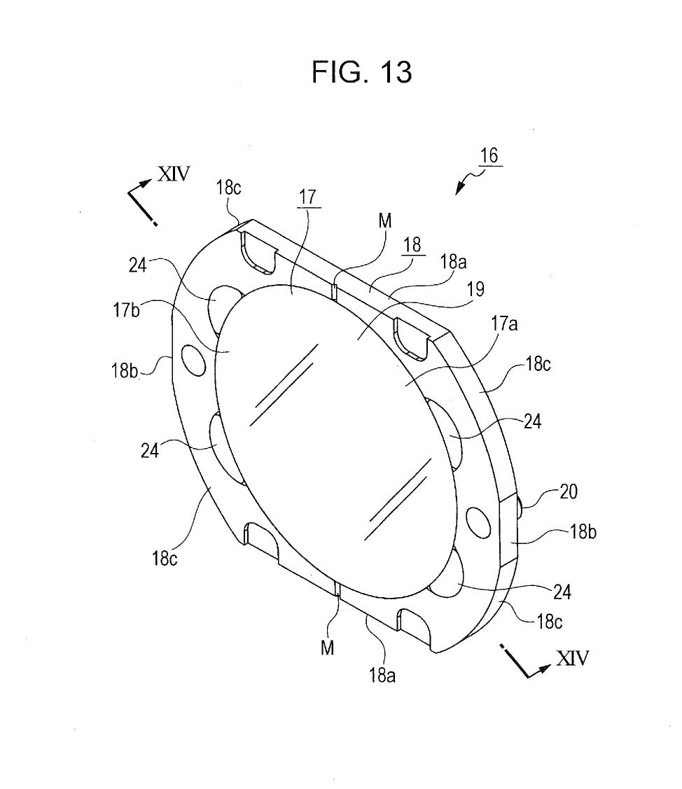

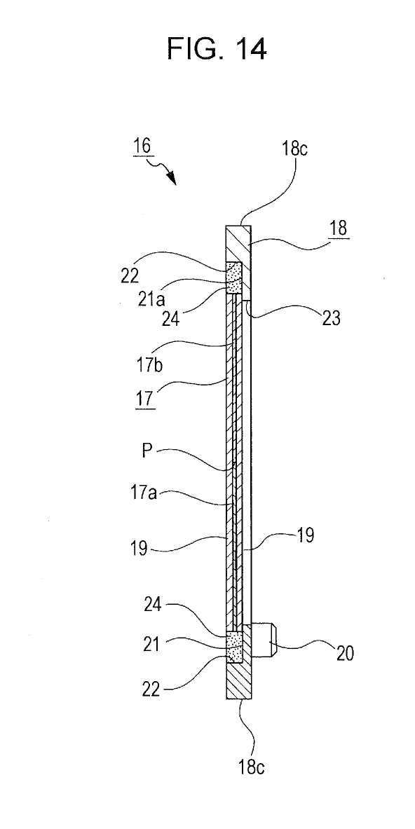

The pupil splitting polarizing element 17 is inserted into the attaching concave portion 21 from the front side, the outer circumferential portion in the rear surface thereof is pressed against the seating surface 21a, and is positioned such that the splitting line P matches the positioning marks M, M which are formed in the element holder 18 (refer to FIG. 13). The pupil splitting polarizing element 17 that is positioned in this manner is fixed to the element holder 18 using adhesive 24, 24, . . . , which the adhesion concave portions 22, 22, . . . are filled with, respectively, and the polarizing element block 16 is configured by the pupil splitting polarizing element 17 being fixed to the element holder 18 (refer to FIGS. 13 and 14).

As described above, since the positioning marks M, M for performing positioning of the splitting line P are formed in the element holder 18, it is possible to easily and reliably position the polarizers 17a, 17b in relation to the element holder 18.

### Attachment of Polarizing Element Block to Scope Holder

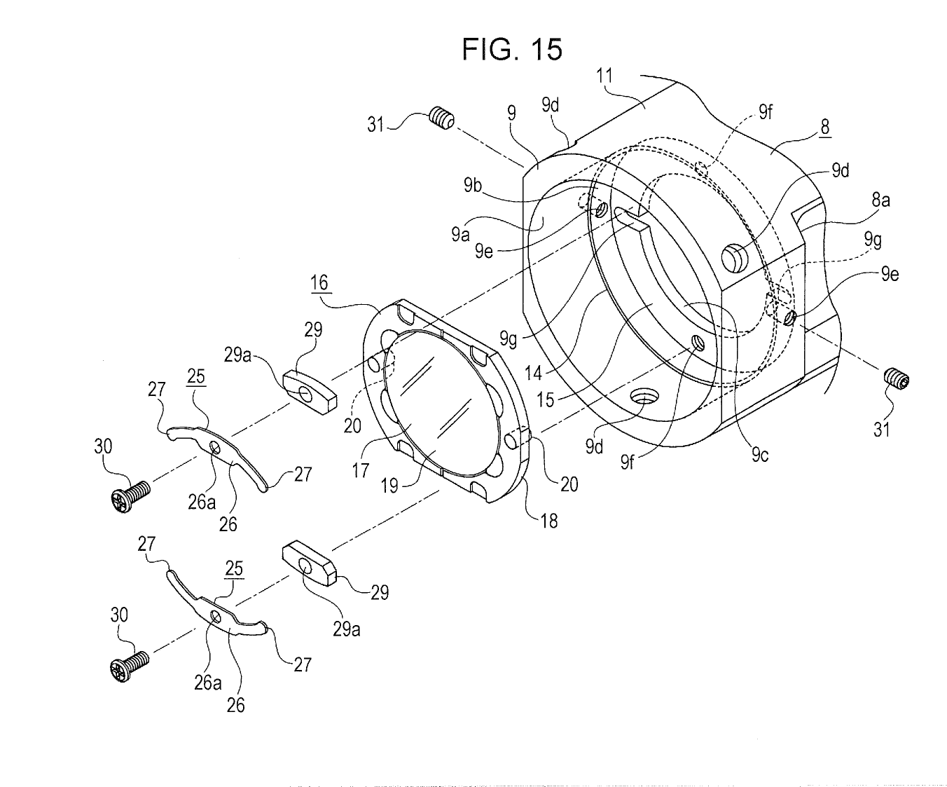

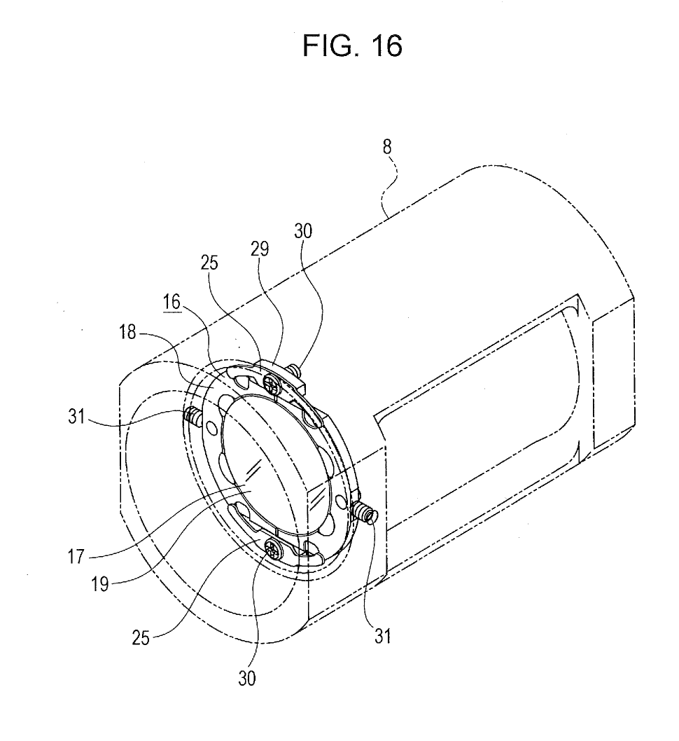

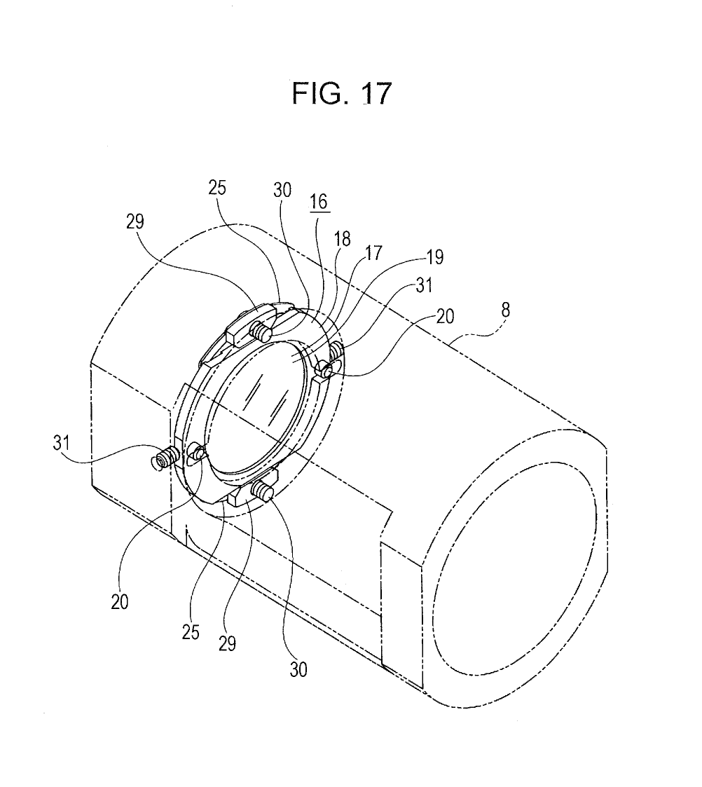

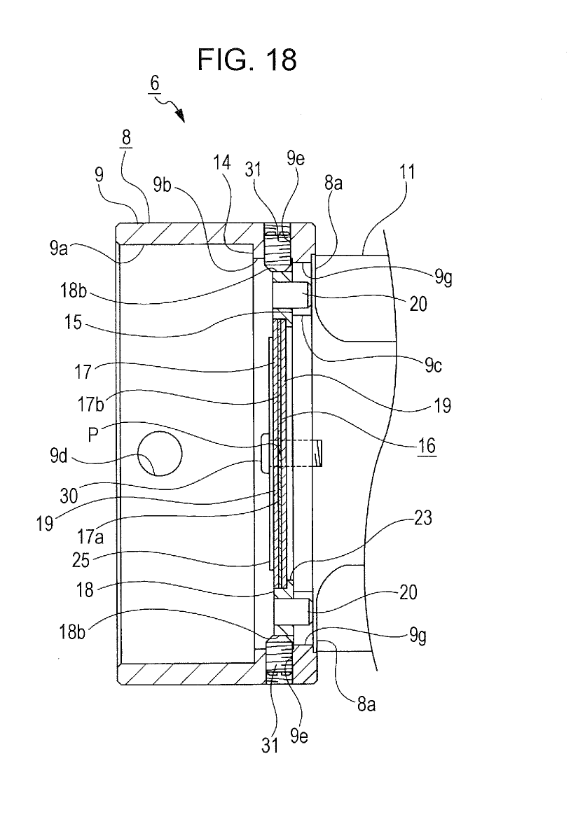

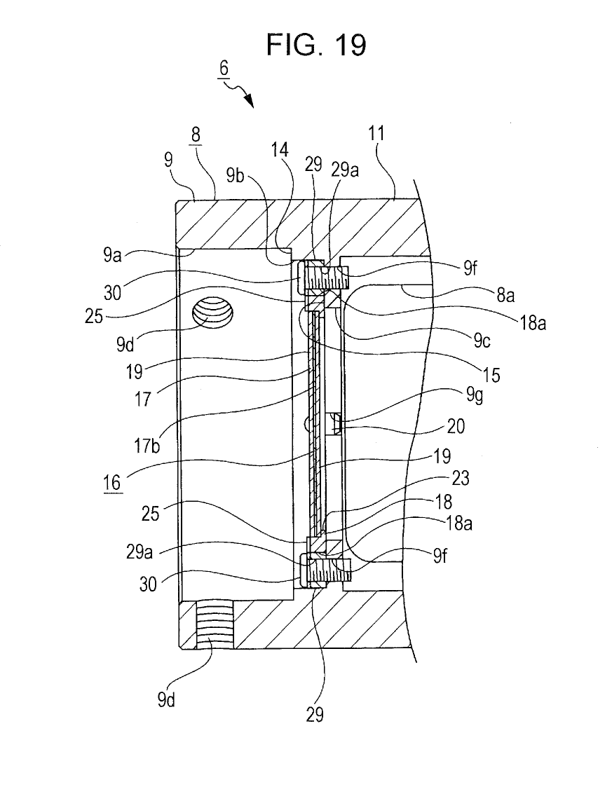

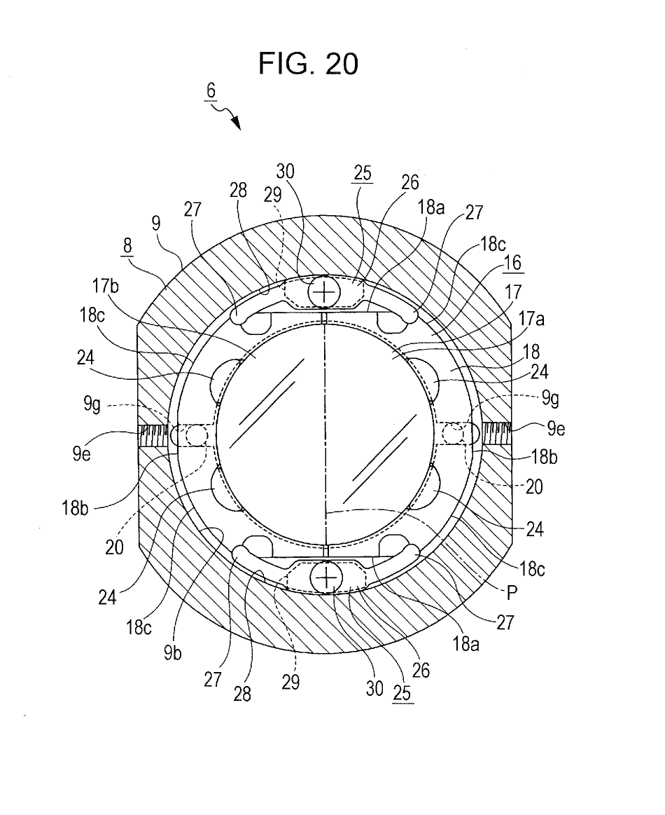

The polarizing element block 16 is attached to the scope holder 8 in a state of being retained by biasing springs 25, 25 (refer to FIGS. 15 to 20). The biasing spring 25 is a plate spring that faces the front and rear directions, for example, and is formed of an attachment target surface portion 26 and retaining arm portions 27, 27 which protrude from approximately the sides of the target surface portion 26, and are formed in an arc shape. An insertion through hole 26a is formed in the attachment target surface portion 26.

The polarizing element block 16 is disposed in the disposition space 9b which is formed in the attaching portion 9 of the scope holder 8, and the positioning pins 20, 20 are inserted into the positioning grooves 9g, 9g, respectively. At this time, since the cutout surfaces 18a, 18a are formed in the element holder 18, voids are formed in both top and bottom end portions of the disposition space 9b. The voids are formed as member disposition spaces 28, 28.

Screw insertion members 29, 29 are disposed in the member disposition spaces 28, 28, respectively. A screw insertion through hole 29a is formed in the screw insertion member 29.

The attachment target surface portions 26, 26 of the biasing springs 25, 25 are pressed against the screw insertion members 29, 29, from the front side, respectively, attaching screws 30, 30 are inserted through the insertion through holes 26a, 26a and the screw insertion members 29, 29, respectively, in order, and the attaching screws 30, 30 are screwed into the screw holes 9f, 9f which are formed in the attaching portion 9 of the scope holder 8. Therefore, the biasing springs 25, 25 are fixed to the attaching portion 9 by the attaching screws 30, 30 via the screw insertion members 29, 29, respectively.

In regard to the polarizing element block 16, in a state in which the biasing springs 25, 25 are each fixed to the attaching portion 9, the top portion and the bottom portion of the element holder 18 are retained from the front by the retaining arm portions 27, 27, . . . of the biasing springs 25, 25, respectively, and the element holder 18 is disposed in the disposition space 9b with the outer circumferential portion thereof being pressed against the retaining surface 15 of the attaching portion 9.

The polarizing element block 16 is pressed against the retaining surface 15 by being retained from the front by the retaining arm portions 27, 27, . . . of the biasing springs 25, 25, and the positioning pins 20, 20 are inserted into the positioning grooves 9g, 9g, respectively; thus, the polarizing element block 16 is capable of moving in the horizontal direction in relation to the scope holder 8. The positioning pins 20, 20 and the positioning grooves 9g, 9g allow the polarizing element block 16 to move in the horizontal direction in relation to the scope holder 8, and function as positioning portions which position the polarizing element block 16 in the horizontal direction in relation to the scope holder 8. Therefore, the horizontal direction, that is, the direction that is perpendicular to both the splitting line P of the pupil splitting polarizing element 17 and the optical axis is set to be the positioning direction.

The pupil splitting polarizing element 17 is positioned in the optical axis direction (the front-rear direction) in relation to the scope holder 8 by the polarizing element block 16 being pressed against the retaining surface 15.

The pupil splitting polarizing element 17 is positioned in the vertical direction and the direction around the optical axis in relation to the scope holder 8 by the positioning pins 20, 20 being inserted into the positioning grooves 9g, 9g, respectively.

In a state in which the element holder 18 is disposed in the disposition space 9b by being pressed against the retaining surface 15, the pupil splitting polarizing element 17 is disposed at the conjugate position of the diaphragm.

In the stereoscopic endoscope 1 that is configured as described above, when substantially afocal light which is emitted from the observation portion 5 of the monocular endoscope 2 is incident on the pupil splitting polarizing element 17, the incident light is polarized by the polarizers 17a, 17b of the element holder 18 in order to generate a right eye image and a left eye image, respectively. The polarized light is polarized by the wire-grid polarizers 12a, 12a, 12b, 12b, . . . of +45° and −45°, respectively, is subjected to photoelectric conversion in the imaging device 12, the right eye image and the left eye image are each generated, and the stereoscopic image is acquired.

As described above, the pupil splitting polarizing element 17 is attached to the attaching portion 9 of the scope holder 8 in a state of being held in the element holder 18. Therefore, it is easy to dispose the pupil splitting polarizing element 17 in the inner portion of the scope holder 8, it is not necessary to grip the pupil splitting polarizing element 17 to dispose the pupil splitting polarizing element 17 on the inner portion of the scope holder 8, the pupil splitting polarizing element 17 is not dirtied or broken, and it is easy to handle the pupil splitting polarizing element 17.

As described above, by disposing the pupil splitting polarizing element 17 in the conjugate position of the diaphragm, the pupil splitting polarizing element 17 is disposed in an optimal position without influencing the optical performance of the imaging optical system, and it is possible to achieve miniaturization in the optical axis direction in addition to securing favorable optical performance of the stereoscopic endoscope 1.

Note that, in the above description an example is given in which the pupil splitting polarizing element 17 is disposed in the conjugate position of the diaphragm; however, the pupil splitting polarizing element 17 may be disposed in the proximity of the conjugate position of the diaphragm.

Disposing the pupil splitting polarizing element 17 in the proximity of the conjugate position of the diaphragm allows the pupil splitting polarizing element 17 to be disposed in an optimal position taking aberration that occurs in the imaging optical system into consideration, and it is possible to achieve miniaturization in the optical axis direction in addition to securing more favorable optical performance of the stereoscopic endoscope 1.

### Positioning Work of Polarizing Element Block

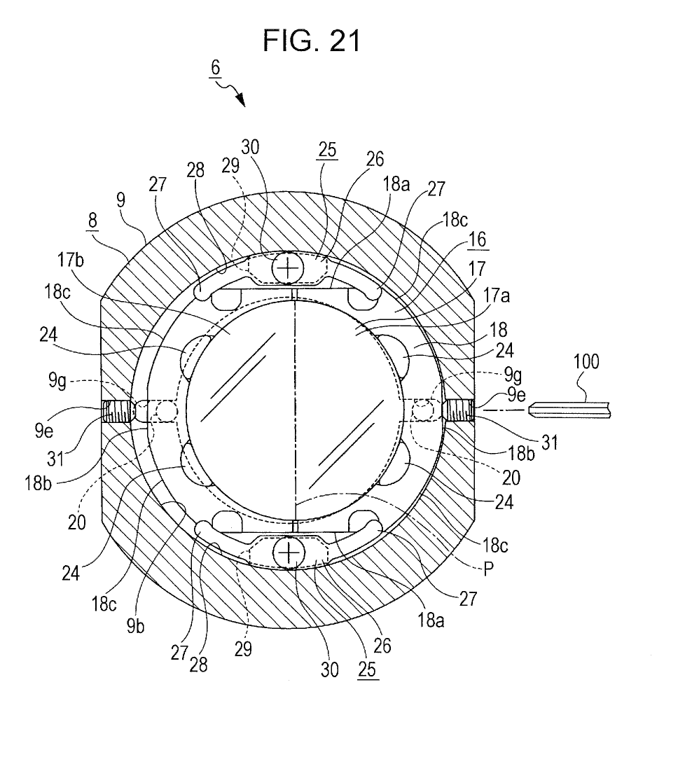

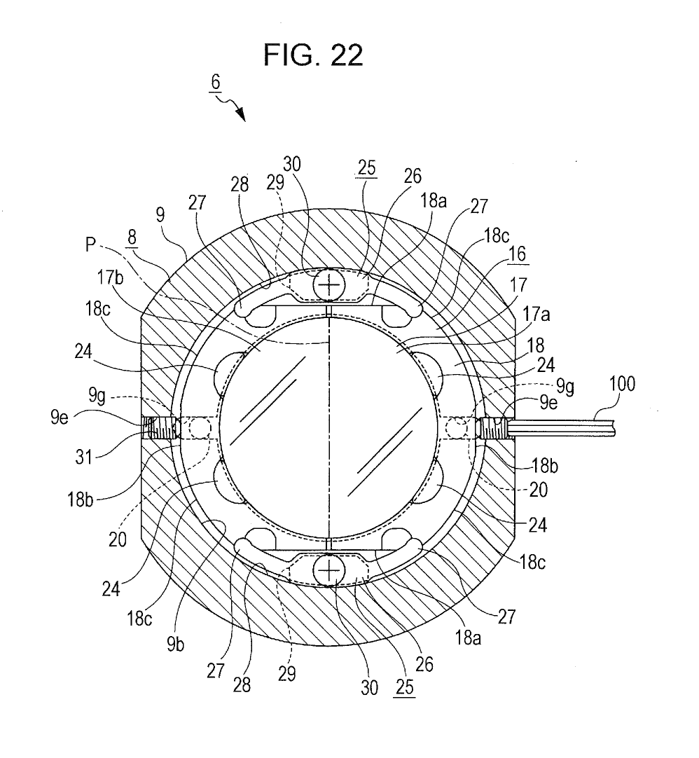

Next, description will be given of the positioning work of the polarizing element block 16 in the horizontal direction in relation to the conjugate position of the diaphragm, or the proximity thereof (refer to FIGS. 21 and 22).

The pupil splitting polarizing element 17 is positioned in the horizontal direction by positioning the polarizing element block 16 in the horizontal direction. The positioning work of the polarizing element block 16 in the horizontal direction in relation to the conjugate position of the diaphragm, or the proximity thereof, is performed in a state in which the polarizing element block 16 is pressed against the retaining surface 15 by being retained from the front by the biasing springs 25, 25.

In the positioning work of the polarizing element block 16 in the horizontal direction, adjusting screws 31, 31 are respectively screwed into the jig insertion holes 9e, 9e that are formed in the attaching portion 9, adjusting jigs 100, 100 such as screwdrivers are inserted into the jig insertion holes 9e, 9e, and the positioning work is performed by causing the adjusting screws 31, 31 to rotate using the adjusting jigs 100, 100.

The distal ends of the adjusting screws 31, 31 respectively make contact with the side surfaces 18b, 18b of the element holder 18. Therefore, due to the adjusting screws 31, 31 being rotated and the position in the horizontal direction changing, the contact positions of the adjusting screws 31, 31 in relation to the side surfaces 18b, 18b change and the polarizing element block 16 is displaced in relation to the scope holder 8; thus the pupil splitting polarizing element 17 is positioned in the horizontal direction in relation to the conjugate position of the diaphragm, or the proximity thereof.

Specifically, in a state in which the polarizing element block 16 is disposed in the disposition space 9b (refer to FIG. 21), one of the adjusting screws 31 is positioned distanced from one of the side surfaces 18b of the element holder 18 in one of the left and right directions; and, in this state, the other adjusting screw 31 is rotated by the adjusting jig 100 approaching the element holder 18. When the other adjusting screw 31 is rotated by the adjusting jig 100, due to the other side surface 18b of the element holder 18 being pressed by the other adjusting screw 31, the positioning pins 20, 20 slide in the positioning grooves 9g, 9g, and the polarizing element block 16 is displaced in one of the left and right directions (refer to FIG. 22). The position of the element holder 18 in the horizontal direction in relation to the scope holder 8 changes due to the polarizing element block 16 being moved in the horizontal direction in this manner, and the element holder 18 is positioned in relation to the conjugate position of the diaphragm, or the proximity thereof.

Note that, as described above, the positioning work of the polarizing element block 16 in relation to the conjugate position of the diaphragm, or the proximity thereof, is performed in a state in which the polarizing element block 16 is retained from the front by the biasing springs 25, 25. Therefore, when the polarizing element block 16 is displaced in the horizontal direction in relation to the scope holder 8, the front surface of the polarizing element block 16 slides in relation to the retaining arm portions 27, 27, . . . of the biasing springs 25, 25.

The positioning work as described above is performed in a state in which the monocular endoscope 2 is joined to the imaging head unit 3. The monocular endoscope 2 is joined to the imaging head unit 3 by the joining portion 5a of the observation portion 5 in the monocular endoscope 2 being inserted, from the front side, into the joining space 9a which is formed in the attaching portion 9 of the scope holder 8, screw members 32, 32, 32 being screwed into the attaching holes 9d, 9d, 9d which are formed in the attaching portion 9, and one end portion of each of the screw members 32, 32, 32 being engaged with the inclined surface 5b of the joining portion 5a.

When the positioning work is performed, in a state in which the monocular endoscope 2 is joined to the imaging head unit 3, a light emitting plate which has a white surface (a diffusing surface) with a uniform luminance is photographed by the stereoscopic endoscope 1, and a video signal of left and right images (a left eye image and a right eye image) is acquired.

When acquiring the video signal, an evaluation value such as that described hereinafter is set in advance for the light intensity distribution difference between the left and right images, and positional adjustment is performed using the positioning work described above such that the evaluation value reaches a minimum based on the acquired video signal. The three points of the screen center, the screen left periphery, and the screen right periphery are selected, for example, as the evaluation points for setting the evaluation value.

The luminance value of the left eye image is set to PLi (where i=1, 2, 3, which indicate the screen center, the screen left periphery, and the screen right periphery, respectively, and this also applies hereinafter), and the luminance value of the right eye image is set to PRi. The luminance value of the left eye image is set to PL′i and the luminance value of the right eye image is set to PR′i in relation to the optimal position in the horizontal direction of the polarizing element block 16, which is determined on the basis of a design value of the imaging optical system which is provided in the stereoscopic endoscope 1.

A center region ΦCoffs=PL1−PR1, a left region ΦLoffs=PL2−PR2, and a right region ΦRoffs=PL3−PR3 are considered as the evaluation value, which is defined from the luminance value between the left and right images. At this time, since it can be considered that ΦCoffs=ΦLoffs=ΦRoffs, it is sufficient to consider only one, ΦCoffs for example, of ΦCoffs, ΦLoffs, or ΦRoffs as the evaluation value. In reality, adjustment balance may be obtained by using an evaluation value obtained by weighting ΦCoffs, ΦLoffs, and ΦRoffs in consideration of aberration of the optical system or the like.

At this time, it is possible to calculate, for the system, an error sensitivity ψCoffs=dΦCoffs/dx of the evaluation value in relation to the displacement in the horizontal direction of the pupil splitting polarizing element 17, and the value thereof is stored in the memory of the system. Note that, dx is the adjustment amount by which the pupil splitting polarizing element 17 is displaced in the horizontal direction during the positioning work.

An evaluation value ΦCoffs in relation to the present position of the pupil splitting polarizing element 17 is calculated from the PLi and the PRi that are acquired from the video signal. If the difference from the design value ΦC′offs (PL′1−PR′1) is used, then dx=(ΦCoffs−ΦC′offs)/ψCoffs is calculated.

When the adjustment amount dx is calculated as described above, the position of the pupil splitting polarizing element 17 is adjusted by rotating the adjusting screws 31, 31 such that the pupil splitting polarizing element 17 moves to the left or the right by dx amount. After adjusting the position of the pupil splitting polarizing element 17 in this manner, the light emitting plate is photographed by the stereoscopic endoscope 1 again and the video signal of the left and right images is acquired. The adjustment amount dx is calculated again in the same manner as described above, based on the acquired value of the video signal, and the position of the pupil splitting polarizing element 17 is adjusted by rotating the adjusting screws 31, 31 according to the calculated dx amount.

Such acquisition of the video signal and position adjustment of the pupil splitting polarizing element 17 based on the calculated adjustment amount dx are performed repeatedly, as necessary, the positional adjustment of the pupil splitting polarizing element 17 is ended when the evaluation value ΦCoffs falls in an acceptable range, and the positioning work in the horizontal direction of the polarizing element block 16 is completed. When the positioning work is completed, the polarizing element block 16 is fixed to the scope holder 8 using an adhesive or the like.

As described above, the positioning work of the pupil splitting polarizing element 17 in relation to the conjugate position of the diaphragm, or the proximity thereof, is performed by the adjusting screws 31, 31, the positions of which in the horizontal direction are changed in relation to the scope holder 8, being rotated. Therefore, the position of the polarizing element block 16 is changed according to the rotation amount of the adjusting screws 31, 31, it is easy to adjust the position of the polarizing element block 16, and it is possible to achieve an improvement in workability in the positioning work of the pupil splitting polarizing element 17.

The adjusting screws 31, 31 are rotated by the adjusting jigs 100, 100, and the jig insertion holes 9e, 9e into which the adjusting jigs 100, 100 are inserted are formed in the scope holder 8; thus, it is possible to perform the positioning work from the outside of the scope holder 8, and it is possible to achieve an improvement in the workability of in the positioning work.

Note that, in the above description an example is given in which the positional adjustment of the pupil splitting polarizing element 17 is performed by the adjusting screws 31, 31; however, the positional adjustment of the pupil splitting polarizing element 17 is not limited to being performed by the adjusting screws 31, 31, and may be performed using an actuator mechanism such as a micro motor or a stepping motor.

### CONCLUSION

As described above, the stereoscopic endoscope 1 is provided with the pupil splitting polarizing element 17 which includes the pair of polarizers 17a, 17b which are disposed to line up along the splitting line P as a boundary, and it is possible to position the pupil splitting polarizing element 17 in relation to the conjugate position of the diaphragm, or the proximity thereof, in the horizontal direction, which is a direction perpendicular to both the splitting line P and the optical axis.

Therefore, since the pupil splitting polarizing element 17 is positioned in the direction in which the polarizers 17a, 17b are lined up, an improvement in the positioning precision of the pair of polarizers 17a, 17b is achieved, and it is possible to acquire a favorable stereoscopic image.

Since the positioning portion for positioning the pupil splitting polarizing element 17 is configured of the positioning pins 20, 20 and the positioning grooves 9g, 9g, it is possible to position the pupil splitting polarizing element 17 reliably using a simple configuration.

The retaining surface 15 is provided on the scope holder 8, and the biasing springs 25, 25 which bias the element holder 18 in the optical axis direction, press the element holder 18 against the retaining surface 15, and position the pupil splitting polarizing element 17 in the optical axis direction are provided.

Therefore, it is easy to position the pupil splitting polarizing element 17 in the optical axis direction, the pupil splitting polarizing element 17 is attached to the scope holder 8 and positioned in the optical axis direction at the same time, and it is possible to achieve an improvement in the workability of the pupil splitting polarizing element 17 in the attachment work in relation to the scope holder 8 and in the positioning work in the optical axis direction.

The cutout surfaces 18a, 18a are formed in the element holder 18, and the member disposition spaces 28, 28, in which the screw insertion members 29, 29 are disposed are formed between the element holder 18 and the scope holder 8, by the cutout surfaces 18a, 18a.

Therefore, the space in the inner portion of the scope holder 8 is used effectively, and it is possible to achieve miniaturization of the stereoscopic endoscope 1.

### Present Technology

The present technology may adopt the following configurations.

(1) A stereoscopic endoscope includes an imaging optical system including a diaphragm which adjusts light intensity and a pupil splitting polarizing element that has a pair of polarizers which are disposed to line up along a splitting line as a boundary; a scope holder in which at least the pupil splitting polarizing element is disposed in an inner portion thereof; and an imaging device on which light is incident via the imaging optical system. Light which is incident on the pupil splitting polarizing element is polarized by the pair of polarizers in order to generate a right eye image and a left eye image, respectively. A direction that is perpendicular to both the splitting line and an optical axis is a positioning direction. Positioning portions are provided to position the pupil splitting polarizing element in relation to a conjugate position of the diaphragm, or a proximity thereof, in the positioning direction.

(2) The stereoscopic endoscope according to (1) further includes an element holder which holds the pupil splitting polarizing element.

(3) In the stereoscopic endoscope according to (2), positioning grooves which extend in the positioning direction are formed in one of the scope holder and the element holder. Positioning pins which extend in an optical axis direction and are supported in the positioning grooves to slide freely are provided on the other of the scope holder or the element holder. The positioning portions are configured of the positioning pins and the positioning grooves. The pupil splitting polarizing element is positioned by changes in relative position of the positioning pins and the positioning grooves.

(4) The stereoscopic endoscope according to (2) or (3) further includes adjusting screws of which a position thereof in the positioning direction is changed when distal ends thereof are pressed against the element holder and the adjusting screws are rotated. The positioning is performed when the adjusting screws are rotated, with positions of the element holder and the pupil splitting polarizing element being changed.

(5) In the stereoscopic endoscope according to (4), the adjusting screw is rotated by an adjusting jig. A jig insertion hole into which the adjusting jig is inserted is formed in the scope holder.

(6) In the stereoscopic endoscope according to any one of (2) to (5), positioning marks for positioning the splitting line during attachment of the pupil splitting polarizing element to the element holder are formed in the element holder.

(7) In the stereoscopic endoscope according to any one of (2) to (6), a retaining surface is formed on the scope holder. Biasing springs which bias the element holder in an optical axis direction, press the element holder against the retaining surface, and position the pupil splitting polarizing element in the optical axis direction.

(8) In the stereoscopic endoscope according to any one of (2) to (7), cutout surfaces are formed on an outer circumference of the element holder. Member disposition spaces, in which predetermined members are disposed, are formed between the element holder and the scope holder by the cutout surfaces.

(9) In the stereoscopic endoscope according to any one of (1) to (8), the pupil splitting polarizing element is disposed in the conjugate position of the diaphragm.

(10) In the stereoscopic endoscope according to any one of (1) to (8), the pupil splitting polarizing element is disposed in a proximity of the conjugate position of the diaphragm.

It should be understood by those skilled in the art that various modifications, combinations, sub-combinations and alterations may occur depending on design requirements and other factors insofar as they are within the scope of the appended claims or the equivalents thereof.

What is claimed is:

1. A stereoscopic endoscope, comprising:
an imaging optical system including a diaphragm which adjusts light intensity and a pupil splitting polarizing element that has a pair of polarizers which are disposed to line up along a splitting line as a boundary;
a scope holder in which at least the pupil splitting polarizing element is disposed in an inner portion thereof; and
an imaging device on which light is incident via the imaging optical system,
wherein light which is incident on the pupil splitting polarizing element is polarized by the pair of polarizers in order to generate a right eye image and a left eye image, respectively,
wherein a direction that is perpendicular to both the splitting line and an optical axis is a positioning direction, and
wherein positioning portions are provided to position the pupil splitting polarizing element in relation to a conjugate position of the diaphragm, or a proximity thereof, in the positioning direction.

2. The stereoscopic endoscope according to claim 1, further comprising:
an element holder which holds the pupil splitting polarizing element.

3. The stereoscopic endoscope according to claim 2,
wherein positioning grooves which extend in the positioning direction are formed in one of the scope holder and the element holder,
wherein positioning pins which extend in an optical axis direction and are supported in the positioning grooves to slide freely are provided on the other of the scope holder or the element holder,
wherein the positioning portions are configured of the positioning pins and the positioning grooves, and
wherein the pupil splitting polarizing element is positioned by changes in relative position of the positioning pins and the positioning grooves.

4. The stereoscopic endoscope according to claim 2, further comprising:
adjusting screws of which a position thereof in the positioning direction is changed when distal ends thereof are pressed against the element holder and the adjusting screws are rotated,
wherein the positioning is performed when the adjusting screws are rotated, with positions of the element holder and the pupil splitting polarizing element being changed.

5. The stereoscopic endoscope according to claim 4,
wherein the adjusting screw is rotated by an adjusting jig, and
wherein a jig insertion hole into which the adjusting jig is inserted is formed in the scope holder.

6. The stereoscopic endoscope according to claim 2,
wherein positioning marks for positioning the splitting line during attachment of the pupil splitting polarizing element to the element holder are formed in the element holder.

7. The stereoscopic endoscope according to claim 2,
wherein a retaining surface is formed on the scope holder, and
wherein biasing springs which bias the element holder in an optical axis direction, press the element holder against the retaining surface, and position the pupil splitting polarizing element in the optical axis direction.

8. The stereoscopic endoscope according to claim 2,
wherein cutout surfaces are formed on an outer circumference of the element holder, and
wherein member disposition spaces, in which predetermined members are disposed, are formed between the element holder and the scope holder by the cutout surfaces.

9. The stereoscopic endoscope according to claim 1,
wherein the pupil splitting polarizing element is disposed in the conjugate position of the diaphragm.

10. The stereoscopic endoscope according to claim 1,
wherein the pupil splitting polarizing element is disposed in a proximity of the conjugate position of the diaphragm.
